(19) United States
(12) Patent Application Publication
(10) Pub. No.: US 2002/0176831 A1
(43) Pub. Date: Nov. 28, 2002

(54) Colored cosmetic composition with novel aesthetics

(75) Inventors: Yinli Wang, Ramsey, NJ (US); Shari Martin, Suffern, NY (US); Jason Rothouse, Nanuet, NY (US); Dawn Lembo, Leonia, NJ (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: AVON PRODUCTS, INC, NEW YORK, NY (US)

(21) Appl. No.: 10/013,851

(22) Filed: Dec. 10, 2001

Related U.S. Application Data
(60) Provisional application No. 60/279,150, filed on Mar. 27, 2001.

(51) Int. Cl. A61K 7/021; A61K 7/025

(52) U.S. Cl. 424/63

(57) ABSTRACT

There are provided colored, cosmetic emulsion compositions and methods of making them. The compositions have a hydrophilic colorant in the aqueous phase of the emulsion and a pearlescent colorant preferably in the oil phase of the emulsion. Upon application to mammalian skin or lips, the compositions of the invention display two or more different colors, have a multi-layered appearance, and display a different color than the color of the composition in its product form.

### RELATED APPLICATION

The present application claims priority in Provisional Application Serial No. 60/279,150 of Yinli Wang et al. filed Mar. 27, 2001, entitled “Colored Cosmetic Composition With Novel Aesthetics.”

### BACKGROUND OF THE INVENTION

1. Field of the Invention

The present invention relates to color cosmetics. More particularly, the present invention preferably relates to a lipstick or other such colored stick or solid and/or semisolid (including creams, gels and viscous liquids) product that throughout the use of the product has a first color prior to use and a different color upon application to mammalian skin or lips. Furthermore, the present invention relates to a lipstick or other colored solid or semisolid colored product that, when applied as a single layer, provides the appearance of multiple layers with each layer being a different shade.

2. Description of the Prior Art

A variety of cosmetic products, including aqueous, non-aqueous, emulsion and powder types, have various inorganic and organic pigments and colorants. Many pigments used commercially do not easily blend into liquid bases, particularly aqueous bases. Various efforts have been made to improve the availability or dispersion of pigments.

Presently, consumers who wish to achieve a two layered look, specifically the effect of a layer of one shade of lipstick covered by a second layer of a different shade of lipstick, e.g. a “frosted” shade, must actually apply multiple layers of multiple shades of lipstick. This is inconvenient for most consumers since achieving the desirable aesthetic appearance requires carrying multiple tubes of lipstick and applying multiple layers of each.

U.S. Pat. No. 5,993,834 to Shah et al. is directed to pigment compositions in which the pigment is treated with a water-dispersible polymer. U.S. Pat. No. 5,853,712 to Langlois is directed to color cosmetic emulsions in which water-dispersible pigments are surface treated with hydrophilic polymers. U.S. Pat. No. 4,648,908 to Kamakura et al. is directed to the coating of water-dispersible pigments using metallic soap. U.S. Pat. No. 4,622,074 to Miyashi et al. provides the surface treatment of pigments with hydrogenated lecithin. U.S. Pat. No. 5,744,129 to Dobbs et al. provides for the use of water-dispersible sulfopolyester and the incorporation of colorant.

The cosmetic industry has a long-standing interest in the development of various shades of color and palettes of ranges of color shades. Development efforts in this area are well known to those skilled in the art of lipstick manufacture. Typically, finely dispersed colorants in oils, as well as pearlescent pigments, are added to the base formulation of the lipstick. In addition, other visual effects can be achieved by spraying or dipping, or by use of other mechanical means, so that a lipstick pomade has glitter or pearlescent colorants. Such commercial products have a thin metallic surface layer, but the majority of the lipstick, i.e., the interior of the bullet, has a different shade of a color. However, the surface layer is easily removed upon use of the lipstick. Once the surface colored layer is removed, not only is the metallic surface appearance gone, but the lipstick no longer provides a multilayered appearance upon application.

In the cosmetics industry, a large number of compositions, methods of manufacture and methods of use, have been developed to provide color characteristics to cosmetics. Cosmetics that contain colorants include, but are not limited to, eye shadow, lipstick, pancake, face stick, eyeliner, foundation, brow pencil, rouge, and the like. A recent trend involves the development of cosmetic compositions that have modifiable or color-varying characteristics. One method of providing color modification is by the addition of stains or colorants that are moisture activated, thus effecting the applied color. However, these generally suffer limitations in that they tend to leave a person's lips extremely dry and they are difficult to remove.

Color changing cosmetic compositions of eye shadow, lipstick, eyeliner, foundation, brow pencil, cream rouge and compact eye shadow involving the use of encapsulated pigments of contrasting color are described in U.S. Pat. No. 4,756,906 to Sweeney. This color changing effect relates to anhydrous products and relies upon encapsulation technology, specifically of pigments and colorants that are added to the anhydrous formulation. This technology has limitations, including cost, timing associated with encapsulating colorants, and gritty unappealing texture from these encapsulates incorporated into a lipstick. Also, most information available in the prior art regarding developing shades of color, shades of color with different effects, or color changing shades, is related to non-emulsion based formulations. In addition, although water-based emulsion lipsticks based upon new technology are becoming increasingly popular, the knowledge of creating novel shades of color and shade ranges of color is limited.

In spite of the various cosmetic products on the market, there remains a need for a solid and/or semi-solid color cosmetic, preferably a lipstick, which displays different colors upon application to a user, multi-dimensional optics, and/or the appearance of multiple layers with each layer being a different shade or color, that is achieved with the application of only a single layer. Also, there is a need for a color cosmetic that provides smooth application in contrast to the gritty texture products provided by the prior art. Further, consumers desire color cosmetics that provide the benefits of water based emulsions, such as increased hydration, in stunning, novel shades.

### SUMMARY OF THE INVENTION

It is an object of the present invention to provide solid or semi-solid color cosmetics that display a multidimensional appearance with only a single layer of application.

It is another object of the present invention to provide such color cosmetics that have a multi-layered appearance upon an application of a single layer on the skin or lips of the user.

It is still another object of the present invention to provide such color cosmetics that have a pearlescence appearance upon an application of a single layer on the skin or lips of the user.

It is yet another object of the present invention to provide cosmetic compositions, preferably lipsticks, that display different colors upon a single application to the user, and each color may differ as compared to the color of the lipstick bullet.

It is a further object of the present invention to provide such color cosmetics that have smooth, rather than gritty, application characteristics.

It is a still further object of the present invention to provide such color cosmetic compositions that last a relatively long period of time.

These and other objects and advantages of the present invention and equivalents thereof, are achieved by the emulsion-based color cosmetic compositions described herein. Emulsion compositions of the present invention have an aqueous phase and an oil phase. The aqueous phase of the emulsion has one or more hydrophilic colorants. The composition also has one or more pearlescent colorants. The one or more pearlescent colorants can be in the oil phase, added in the formed emulsion, or one or more pearlescent colorants can be in the oil phase and one or more other pearlescent colorants added to the formed emulsion. In a preferred embodiment, one or more pearlescent colorants are in the oil phase and one or more additional pearlescent colorants are added after formation of the emulsion to achieve desired effects including the desired layering effect.

The present invention provides a method for the production of colored cosmetic compositions. The method includes mixing an aqueous phase having one or more hydrophilic colorants with an oil phase, preferably, having one or more pearlescent colorants for a period of time sufficient to form an emulsion. In the preferred embodiment, one or more additional pearlescent colorant of the same or different pigmentation from that in the oil phase are added to the composition after the formation of the emulsion to achieve the desired effects.

### DESCRIPTION OF THE DRAWINGS

FIG. 1 is a schematic diagram of the cross section of a preferred embodiment, a lipstick bullet, of an emulsion composition of the present invention showing colorant in both the oil and water phases; and

FIG. 2 is a diagram of process steps in the manufacture of the preferred embodiment illustrated in FIG. 1.

### DETAILED DESCRIPTION OF THE INVENTION

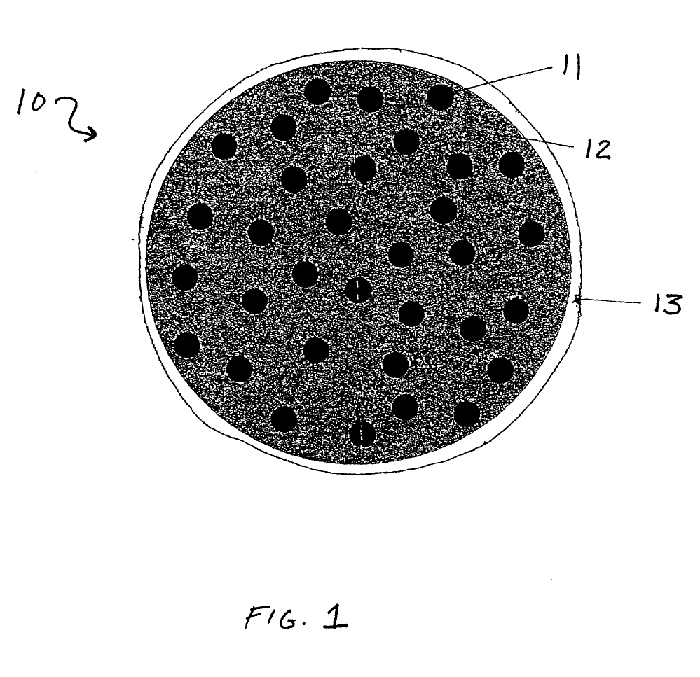

The present invention provides a variety of colored emulsion-based cosmetic compositions useful in solid and/or semi-solid forms (including creams, gels and viscous liquids). Such compositions are preferably lipsticks, but also include eye shadows, eyeliners, foundations, brow pencils, face sticks, pancakes, and other facial cosmetic products. Referring to the figures and particularly to FIG. 1, a cross section of a preferred embodiment of the present invention, a lipstick, is generally represented by reference numeral 10. The lipstick bullet 10 is a water-based emulsion lipstick.

Preferably, the lipstick bullet 10 is a water-in-oil emulsion. This emulsion has a water or internal phase 11 and an oil or continuous phase 12. The water (aqueous) or internal phase 11 of the emulsion has one or more hydrophilic colorants or a color dispersion. In a preferred embodiment, the oil or continuous phase 12 has one or more pearlescent colorants 22, and the emulsified composite lipstick bullet has one or more additional pearlescent colorants 13 that could be the same or different than pearlescent colorants 22. Whether one or more pearlescent colorants are in the oil phase 12, added to the formed emulsion, or both, the result is a bullet of pearlescent and layered appearance. However, the most desired appearance is achieved with one or more pearlescent colorants 13/22 in the oil phase 12 and added to the formed emulsion. A less preferred embodiment, has one or more pearlescent colorants 13 only in the oil phase, and the least preferred embodiment has one or more pearlescent colorants 22 only added to the formed emulsion.

During the course of research in the area of water-based lipstick formulation, it was discovered that by utilizing water-dispersible colorants, preferably hydrophilic colorants, novel color shades and visual effects could be created.

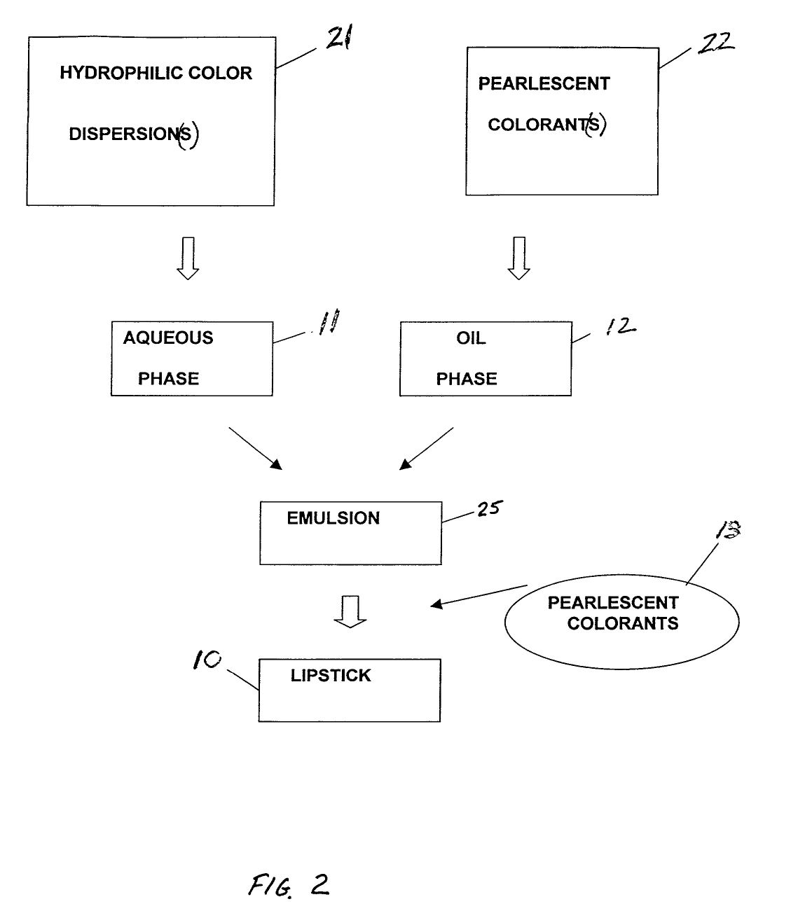

Referring to FIG. 2, the manufacture of an emulsion-based lipstick is illustrated. To create the novel visual effects of the present compositions, aqueous phase 11 of an emulsion based lipstick has one or more hydrophilic colorants 21. Preferably, these colorants are hydrophilic/water-dispersible or have been modified to be hydrophilic/water-dispersible. These colorants include, but are not limited to, FD&C Yellow 5, FD&C Yellow 5 Al Lake, FD&C Red 40, FD&C Yellow 6, FD&C Blue 1, FD&C Blue 1 Al Lake, D&C Yellow 10, D&C Orange 5, D&C Red 6, D&C Red 7, D&C Red 21, D&C Red 22, D&C Red 27, D&C Red 27 Al Lake, D&C Red 28, D&C Red 30, D&C Red 33, D&C Red 34, D&C Red 36, D&C Red 34 Ca Lake, D&C Red 6 Ba Lake, D&C Red 7Ca Lake, titanium dioxide, iron oxides, red iron oxide, green iron oxide, black iron oxide, and ultramarine colorants. Examples of other suitable hydrophilic colorants are set forth in U.S. Pat. Nos. 5,863,459 and 5,904,878, the disclosures of which are incorporated herein by reference. In addition a water-insoluble pigment may be added, preferably to the oil phase. Such pigments include inorganic, organic or any combination thereof that are insoluble or sparingly soluble in water.

In compositions of the present invention, the one or more hydrophilic colorants total about 0.1 weight percent (wt %) to about 50 wt % of the total weight of the composition. More preferably, the amount of the hydrophilic colorant(s) is about 1 wt % to about 15 wt %. Optimally, the hydrophilic colorant is about 5 wt % to about 12 wt % of the total weight of the composition.

As discussed above, the compositions of the present invention must have one or more pearlescent colorants. However, the one or more pearlescent colorants can be in the oil phase, added to the composition after the emulsion is formed, or both. It is preferable that the one or more pearlescent colorants be added in the oil phase since the desired effects are achieved even better than if the one or more pearlescent colorants are simply added into the formed emulsion. In the most preferred compositions of the present invention, at least one pearlescent colorant is in the oil phase and one or more additional pearlescent colorants are also added to the formed emulsion (i.e., external to the formed emulsion) in order to achieve the most desired effects.

Referring to the most preferred embodiment that is shown in the drawings, the oil phase 12 of the present compositions has one or more pearlescent colorants 22. As discussed before, aqueous phase 11 with hydrophilic colorant(s) 21 and the oil phase 12 with pearlescent colorant(s) 22 are mixed together to form an emulsion 25. After the emulsion 25 is formed, one or more pearlescent colorants 13 are preferably added to emulsion 25 giving the bullet its desired surface effect, namely a pearlescent outer appearance, and a layering effect.

For use in the present invention, pearlescent colorants 13/22 are those that typically have been added to provide metallic type color in cosmetic products. Such pearlescent colorants 13/22 include, but are not limited to, one or more titanated micas, titanium dioxide coated micas, micas, bismuth oxychlorides, bismuth oxychloride coated with colorants, boron nitrides, copper powders, and bronze powders.

Pearlescent colorants 13/22 in the oil phase, formed emulsion or both, are present in a total amount about 0.1 wt % to about 50 wt % of the total weight of the composition. The pearlescent colorants are preferably present in a total amount about 5 wt % to about 30 wt %, and more preferably about 8 wt % to about 20 wt %, of the total weight of the composition.

The present emulsion cosmetic composition provides the benefits of a multi-layered effect but in a single application. In particular, the composition has a base formula to deposit color and a colorant system to allow the pearl to sit on the pigments.

The emulsion-based compositions of the present invention are water-in-oil, oil-in-water or triple emulsions. Preferably, the compositions are water-in-oil emulsions.

The aqueous phase 11 of the present compositions may have a pharmaceutically or cosmetically acceptable material. A typical water phase of the present invention used in an emulsion has one or more of the following ingredients: emulsifiers, emollients, colorants, film formers, water, and any other ingredient known in the art that can be used in a water phase of an emulsion.

Suitable components for the oil phase of the present compositions include, but are not limited to, one or more waxes, oils, pigments, emulsifiers, film formers, fillers, preservatives, and fragrances.

Suitable emulsifiers vary depending on the selection of waxes, oils, and the percentage of water in the final formula. The emulsifiers can be selected by those skilled in the art based on the desired end point.

As is known in the art, with preferred lipstick embodiments of the present invention, the lipstick bullet is in lipstick form when it is presented in a lipstick package. The cosmetic compositions of the present invention provide several desired effects, namely several different novel aesthetics upon application.

The first novel aesthetic appearance is the dual layer appearance of a first layer of cream or matte lipstick superimposed by a second layer of pearl or frosted or metallic lipstick, in a single layer of a present lipstick. The present invention removes the burden of requiring the consumer to purchase, carry and apply two products to obtain a dual layer appearance.

The second novel aesthetic appearance is that upon application, the single layer provides not only a multi-dimensional look, but also at least two (or more) distinct colors depending on the vantage point of the person viewing the user.

A third novel aesthetic appearance provided by a lipstick of the present composition is the contrasting color/shade provided by the lipstick bullet when compared to the color/shade of the composition on the skin or lips of the user. For example, the lipstick bullet may be a tawny pearlescent gold, yet upon application the lipstick has a reddish undertone with a gold frost overtone. The colors that can be achieved vary depending on the desired undertone shade and top tone shade. These include, but are not limited to, brown/silver, brown/gold, pink/silver, pink/gold, plum/green, and plum/blue. Other contrasting shades can be achieved depending on the selection of pigment dispersions and pearlescent colorants.

The present invention is further illustrated by the following non-limiting example.

| Component | wt % Range |
|---|---|
| Castor Oil | 15-45 |
| Diisostearyl Fumarate | 5-10 |
| Lanolin | 5-10 |
| Cetyl Lactate | 5-10 |
| Wax (e.g. candilla wax, micro- | 0.5-50 |
| crystalline wax, carnauba wax, | |
| paraffin wax, and synthetic waxes) | |
| Aqueous Phase Emulsifying | 0.5-5 |
| Agent (PEG-8) | |
| Preservative | 0.1-1 |
| Sodium Silicoaluminate | 0.1-1 |
| Demineralized Water | q.s. |
| Hydrophilic Colorants | 0.1-50 |
| Oil Phase Emulsifying Agent | 0.5-5 |
| (e.g. polyglyceryl isostearate) | |
| Pearlescent Colorants | 0.1-50 |
| Fragrance | 0.1-1 |

In the method of making the preferred compositions of the present invention, demineralized water, the aqueous emulsifying agent, and the hydrophilic colorants are premixed in a vessel. To a separate vessel, the wax, oil and oil emulsifying agent are added and heated to 170 to 190 degrees F. with mixing. Once the temperature is constant and the waxes and oils are well mixed, pearlescent colorant is added to the mixture. Mixing continues until all of the pearls are evenly dispersed. Then the premixed colorants, water, and emulsifier are added to the oil/wax/pearlescent mixture. Mixing continues for 10 to 60 minutes and then additional pearls are added slowly, one color at a time, to this mixture. After the selected pearls are added, fragrance is added prior to forming the lipstick.

Although the present invention describes in detail certain embodiments, it is understood that variations and modifications may exist that are known to those skilled in the art but, nonetheless, fall within the scope of the present invention. Accordingly, the present invention is intended to encompass all such alternatives, modification and variations that are within the scope of the invention as set forth in the following claims.

What is claimed is

1. A colored cosmetic composition comprising:
an emulsion formed of an aqueous phase and an oil phase, said aqueous phase having at least one hydrophilic colorant; and
at least one pearlescent colorant, said at least one pearlescent colorant being present in the oil phase and/or external to the formed emulsion,
wherein the composition has a multi-layered and/or multi-dimensional appearance upon application of a single layer of the composition.

2. The composition according to claim 1, wherein the composition has at least two different visual effects.

3. The composition according to claim 2, wherein the two different visual effects are two distinct colors.

4. The composition according to claim 1, wherein the composition is an emulsion selected from the group consisting of oil-in-water, water-in-oil, and triple emulsion.

5. The composition according to claim 1, wherein said at least one pearlescent colorant is in said oil phase.

6. The composition according to claim 5, further comprising one or more additional pearlescent colorants external to the formed emulsion.

7. The composition according to claim 1, wherein the composition is a form selected from the group consisting of a solid and a semi-solid.

8. The composition according to claim 1, wherein said at least one pearlescent colorant is external to the formed emulsion.

9. The composition according to claim 1, wherein the composition is a product form selected from the group consisting of eye shadow, lipstick, pancake, face stick, eyeliner, foundation, brow pencil, and rouge.

10. The composition according to claim 1, wherein the composition is a lipstick.

11. The composition according to claim 1, wherein said at least one pearlescent colorant is present in an amount about 0.1 wt % to about 50 wt % of the total weight of the composition.

12. The composition according to claim 1, wherein said at least one hydrophilic colorant is present in an amount about 0.1 wt % to about 50 wt % of the total weight of the composition.

13. The composition according to claim 1, wherein said at least one pearlescent colorant is present in an amount about 5 wt % to about 30 wt % of the total weight of the composition.

14. The composition according to claim 1, wherein said at least one hydrophilic colorant is present in an amount about 1 wt % to about 15 wt % of the total weight of the composition.

15. The composition according to claim 1, wherein the composition is a water-in-oil emulsion.

16. The composition according to claim 1, wherein said at least one hydrophilic colorant is selected from the group consisting of FD&C Yellow 5, FD&C Yellow 5 Al Lake, FD&C Red 40, FD&C Yellow 6, FD&C Blue 1, FD&C Blue 1 Al Lake, D&C Yellow 10, D&C Orange 5, D&C Red 6, D&C Red 7, D&C Red 21, D&C Red 22, D&C Red 27, D&C Red 27 Al Lake, D&C Red 28, D&C Red 30, D&C Red 33, D&C Red 34, D&C Red 36, D&C Red 34 Ca Lake, D&C Red 6 Ba Lake, D&C Red 7Ca Lake, titanium dioxide, iron oxides, red iron oxide, black iron oxide, green iron oxide, and ultramarine colorants, and any combinations thereof.

17. The composition according to claim 16, wherein said at least one hydrophilic colorant is water dispersible.

18. The composition according to claim 1, wherein said at least one pearlescent colorant is selected from the group consisting of titanated mica, titanium dioxide coated mica, mica, bismuth oxychloride, bismuth oxychloride coated with colorants, boron nitride, copper powder, bronze powder, and any combinations thereof.

19. A method of imparting a multi-layered and/or multi-dimensional color appearance to skin and/or lips comprising topically applying to the skin and/or lips the composition of claim 1.

20. The method according to claim 19, wherein the composition is applied as a single layer.

21. The method according to claim 20, wherein said at least one pearlescent colorant is selected from the group consisting of titanated mica, titanium dioxide coated mica, mica, bismuth oxychloride, bismuth oxychloride coated with colorants, boron nitride, copper powder, bronze powder, and any combinations thereof.

22. The method according to claim 19, wherein said at least one hydrophilic colorant is selected from the group consisting of FD&C Yellow 5, FD&C Yellow 5 Al Lake, FD&C Red 40, FD&C Yellow 6, FD&C Blue 1, FD&C Blue 1 Al Lake, D&C Yellow 10, D&C Orange 5, D&C Red 6, D&C Red 7, D&C Red 21, D&C Red 22, D&C Red 27, D&C Red 27 Al Lake, D&C Red 28, D&C Red 30, D&C Red 33, D&C Red 34, D&C Red 36, D&C Red 34 Ca Lake, D&C Red 6 Ba Lake, D&C Red 7Ca Lake, titanium dioxides, iron oxides, red iron oxide, black iron oxide, green iron oxide, and ultramarine colorants, and any combinations thereof.

23. The method according to claim 19, wherein said at least one hydrophilic colorant is present in an amount about 0.1 wt % to about 5 0 wt % of the total weight of the composition.

24. The method according to claim 19, wherein said at least one pearlescent colorant is present in an amount about 0.1 wt % to about 50 wt % of the total weight of the composition.

25. The method according to claim 19, wherein said at least one hydrophilic colorant is present in an amount about 1 wt % to about 15 wt % of the total weight of the composition.

26. The method according to claim 19, wherein said at least one pearlescent colorant is present in an amount about 5 wt % to about 30 wt % of the total weight of the composition.

27. The method according to claim 19, wherein the composition is a form selected from the group consisting of a solid and a semi-solid.

28. The method according to claim 19, wherein the composition is selected from the group consisting of eye shadow, lipstick, pancake, face stick, foundation, eyeliner, brow pencil, and rouge.

29. The method according to claim 18, wherein the composition is a lipstick.

30. A colored cosmetic composition comprising:
an emulsion formed of an aqueous phase and an oil phase, said aqueous phase having at least one hydrophilic colorant; and
at least one pearlescent colorant, said at least one pearlescent colorant being present in the oil phase and/or external to the formed emulsion,
wherein upon application to the lips of a mammal the composition provides at least two different visual effects.

31. The composition according to claim 30 , wherein said at least one additional pearlescent colorant is in the oil phase.

32. The composition according to claim 31, further comprising one or more additional pearlescent colorants external to the formed emulsion.

33. The composition according to claim 30, wherein the composition is an emulsion selected from the group consisting of oil-in-water, water-in-oil, and triple emulsion.

34. A semi-solid or solid cosmetic comprising:
an emulsion formed of an aqueous phase and an oil phase, said aqueous phase having at least one hydrophilic colorant, said oil phase having at least one pearlescent colorant,
wherein the cosmetic has a first color upon visual inspection, but delivers a second color upon application to skin or lips.
wherein the cosmetic has a first color upon visual inspection, but delivers a second color upon application to skin or lips.

35. The composition of claim 34, further comprising one or more additional pearlescent colorants external to the formed emulsion.

36. The cosmetic of claim 34, wherein the cosmetic is a lipstick bullet.

37. A process for making a colored cosmetic composition comprising:
forming an emulsion having an aqueous phase and an oil phase;
adding at least one hydrophilic colorant to the aqueous phase; and
adding at least one pearlescent colorant to the oil phase and/or to the formed emulsion.

38. A colored cosmetic composition made by the process of claim 37.
